(19) United States
(12) Patent Application Publication
(10) Pub. No.: US 2005/0084507 A1
(43) Pub. Date: Apr. 21, 2005

(54) Liquid preparations and surfactant/solvent systems

(76) Inventors: Jochen Wurtz, Bingen am Rhein (DE); Thomas Maier, Hofheim (DE); Gerhard Schnabel, Elsenfeld (DE); Gerhard Johann, Idstein (DE)

Correspondence Address: FROMMER LAWRENCE & HAUG, 745 FIFTH AVENUE- 10TH FL., NEW YORK, NY, 10151, US

(21) Appl. No.: 10/985,606

(22) Filed: Nov. 10, 2004

Related U.S. Application Data
(63) Continuation of application No. 09/937,166, filed on Apr. 29, 2002, now abandoned.

(30) Foreign Application Priority Data
Mar. 23, 1999 (DE) 19913036.1

(51) Int. Cl. A61K 9/00

(52) U.S. Cl. 424/400

(57) ABSTRACT

Surfactant/solvent systems for liquid organic formulations (preparations) which comprise one or more aromatic-based surfactants and one or more completely esterified organic phosphates and/or phosphonates which are as polar as possible, but are at the same time water-insoluble or soluble in water to 5 g/l, as solvents, are suitable to be used for the preparation of emulsifiable concentrates and corresponding liquid preparations derived therefrom, such as aqueous spray mixtures.

The invention relates to the field of combinations of surfactants and solvents (surfactant/solvent systems) for liquid preparations (also called formulations). The invention preferably relates here to surfactant/solvent systems for single-phase formulations of one or more pesticide active ingredients where none of the active ingredients is readily soluble in water, each active ingredient preferably having a solubility of 5 grams per liter (g/l) or less than 5 g/l in water. In particular, the invention relates to emulsifiable concentrates (EC) based on organic solvents and pesticidal, e.g. herbicidal, active ingredients of varying polarity, specifically emulsifiable concentrates which comprise one or more active ingredients from the group desmedipham, phenmedipham, ethofumesate and herbicides which are of a similar type with regard to their physical and application properties, for example herbicides from the series of phenoxyphenoxypropionates or heteroarylphenoxypropionates.

In general, active ingredients are not used as pure substances, but, depending on the field of application and the undesired physical nature of the application form, in combination with certain auxiliaries, i.e. they are “formulated”. Frequently, such formulations comprise combinations of different active ingredients instead of individual active ingredients in order to jointly utilize the properties of the individual active ingredients upon application, or else because the individual active ingredients are synergistic in combination, i.e. produce superadditive increases in activity.

Independently of the type of formulation and of whether the formulations comprise one or more active ingredients, the aim in the agricultural sector is, in particular, to achieve the highest possible active ingredient concentration (“loading”) of the formulation in question since a high concentration of the active ingredients permits a reduction in the volumes to be applied and consequently material savings with regard to the auxiliaries applied, and also results in savings with regard to packaging and transportation. For this reason, high-concentration stable formulations and coformulations with environmentally friendly auxiliaries are of fundamental interest.

In principle, active ingredients can be formulated in various ways, depending on which biological and/or chemicophysical parameters are specified. In general, examples of suitable possible formulations are wettable powders (WP), oil-in-water emulsions (OW) and water-in-oil emulsions (EO), suspensions (SC), suspoemulsions (SE), emulsifiable concentrates (EC) or else granules for soil application or scattering, or water-dispersible granules (WG). Said formulation types are known in principle and are described, for example, in Winnacker-Küchler, “Chemische Technologie” [Chemical Engineering], Volume 7, C. Hauser-Verlag, Munich, 4th edition 1986; van Valkenburg, “Pesticide Formulations”, Marcel-Dekker N.Y., 1973; K. Martens, “Spray Drying Handbook”, 3rd edition, 1979, G. Goodwin Ltd., London.

If the active ingredients to be formulated are of low polarity, e.g. compounds which are not salt-like or which contain predominantly hydrophobic radicals and are therefore virtually insoluble in water, the possible formulations are naturally limited. This applies, for example, to herbicidal active ingredients such as desmedipham (DMP) and phenmedipham (PMP), which belong to the group of biscarbamates and whose solubility in water is 7 mg/l and 4.7 mg/l, respectively. Similar behavior is exhibited by herbicidal active ingredients from the group of sulfonates, such as, for example, ethofumesate (solubility in water: 50 mg/l) or benfuresate (solubility in water: 261 mg/l).

Liquid formulations of herbicides of the type given above are already known. Thus, WO-A-85/01286, for example, describes liquid formulations which comprise PMP and/or metamitron. The solvents mentioned in this connection are esters of polyalcohols, ethers, ketones, water-insoluble alcohols, (poly)glycols and oils of vegetable, but also mineral origin, and suitable emulsifiers given for the described liquid formulations are only generally nonionogenic, but also ampholytic, cationic or anionic surfactants.

As an alternative to solvent-based emulsifiable concentrates, water-containing suspension concentrates (SC) or suspoemulsions (SE) are inter alia suitable for the abovementioned active ingredients. Such formulations are described in WO-A-95/23505, EP-A-0637910 and WO-A-92/09195.

In contrast to the thermodynamically stable emulsifiable concentrates, which are notable for their theoretically unlimited storage stability, suspensions, including macroemulsions, are only kinetically stable, i.e. after a period which varies in individual cases, phase separation and thus “breakdown” of the formulation has to be accepted in the case of the latter. In addition, suspensions have the disadvantage compared with emulsifiable concentrates that in the concentrate only some, usually only a small fraction, of the active ingredient or active ingredient mixture used is present in dissolved form. Upon dilution of the suspension with water to prepare the spray mixture, dissolution of the fractions undissolved in the concentrate is in most cases not achieved or not achieved completely, i.e. the spray mixture is still a suspension. As numerous biological experiments have shown, particularly in the case of the active ingredients DMP, PMP and ethofumesate, it is, however, in most cases advantageous if the active ingredients are present as far as possible in the dissolved state in the spray mixture as well: accordingly, the finer the active ingredients are dispersed in the spray mixture, the better the efficiency of the emulsion.

In addition, a very fine distribution of the active ingredients in the spray mixture leads to application advantages, e.g. reduced danger of blockage for the spray nozzles, reduced cleaning costs, etc.

Moreover, in contrast to suspensions, which require grinding of the active ingredient or of the active ingredients, emulsifiable concentrates can advantageously be prepared with a very small input of energy and using technically simple stirring tools, i.e. even during the preparation, advantages arise over said suspensions as a result of a saving in energy costs.

In order to keep the largest possible fraction of active ingredient(s) in solution in the spray mixture as well, the object in the present case is therefore to find liquid preparations with water-insoluble solvents whose polarity permits high concentrations of active ingredients. There is, however, no correlation between the polarity of the solvent and the polarity of the active ingredient(s), which can be used to predict suitable solvents as certain active ingredients. To answer the question as to whether suitable solvents of this type can be found in an individual case or not often requires a laboriously high number of experiments and even then sometimes remains open.

FR-A-2597720, FR-A-2599593 and BE-A-904874 have already described emulsifiable concentrates which, in deviation from the publications cited above, in conjunction with (at least) one herbicide of the biscarbamate type (in particular PMP or DMP) comprise a solvent combination of tributyl phosphate and a water-miscible solvent, such as, in particular, N-methylpyrrolidone (NMP).

Furthermore, EP-A-0328217 describes emulsifiable concentrates which comprise ethofumesate and, as solvent, tributyl phosphate. A disadvantage of the latter type of formulation is the use of tributyl phosphate because it is regarded as a hazardous chemical (cf. e.g. Chemikaliengesetz [German Chemicals Act]). Although this does not make impossible or prohibit the use of tributyl phosphate, the use is, however, generally associated with conditions or is generally problematical.

In addition to the purely formulation object of providing a stable concentrated liquid formulation which, upon dilution with water, gives spray mixtures with favorable physical and performance properties, the object is additionally preferably to provide liquid formulations with biologically favorable properties. The auxiliaries to be used for the liquid formulations should therefore be able to be used widely with regard to the biological properties, and support or as far as possible not impair the properties of the active ingredients used.

Furthermore, it is known that the biological activity of some pesticidal active ingredients can be increased in some cases by low molecular weight organic compounds. Thus, according to BE-A-597284, esters or partial esters based on orthophosphoric acid and alkyl-, aryl-, alkylaryl-, cycloalkyl- and/or heterocycle-based alcohols are suitable for increasing the effect of herbicides, for example of herbicidal phenylurea derivatives such as monuron, azoles, such as amitrol, triazines such as simazine and propionic acid derivatives, such as dalapon. The phosphoric esters specifically described here as auxiliaries include only relatively nonpolar or completely water-soluble phosphoric esters which are not particularly suitable for the preparation of emulsifiable concentrates. In addition, the active ingredients preferred within the scope of the objective, such as biscarbamates (phen- and desmedipham) or sulfonates (ethofumesate) are not mentioned in this publication.

DE-A-2914164 describes synergistic effects which arise in the case of herbicides with a desiccant action on crop plants, i.e. for example herbicides from the group of phenylureas (e.g. metoxuron, diuron) or triazines (e.g. atrazine, simazine), if they are combined with solvents as are used in the metallurgical industry for metal extraction or as plasticizers for polymers. It is not clear from the publication which of the generally specified solvents are suitable for the preparation of emulsifiable concentrates and liquid preparations preparable therefrom.

Surprisingly, it has now been found that certain surfactant/solvent systems are suitable in a particular manner to be used for the preparation of emulsifiable concentrates and corresponding liquid preparations derived therefrom, such as aqueous spray mixtures.

The invention provides surfactant/solvent systems for liquid organic formulations (preparations), characterized in that they comprise
one or more aromatic-based surfactants and
one or more completely esterified organic phosphates and/or phosphonates which are as polar as possible, but which at the same time are water-insoluble or are soluble in water up to 5 g/l, preferably up to 3 g/l, in particular soluble in water up to 2 g/l, as solvent.
   (=surfactant/solvent system according to the invention).

The invention also provides liquid formulations, in particular herbicidal formulations, which comprise
(a) one or more water-insoluble active ingredients,
(b) the surfactant/solvent system according to the invention (=component mixture (b)),
(c) optionally further organic solvents,
(d) optionally further surfactants and/or polymers and
(e) optionally water.

Aromatic-based surfactants which can be used according to the invention are, for example, surface-active benzenes or phenols substituted by one or more alkyl groups and subsequently derivatized and which are soluble in the solvent phase and emulsify said phase, together with the active ingredients dissolved therein, upon dilution with water (to give the spray mixture).

Examples of such surfactants are:
b1.1) phenols, phenyl (C₁-C₄)alkyl ethers or (poly)alkoxylated phenols [=phenol (poly)alkylene glycol elthers], for example having 1 to 50 alkyleneoxy units in the (poly)alkyleneoxy moiety, where the alkylene moiety preferably has in each case 1 to 4 carbon atoms, preferably phenyl reacted with 3 to 10 mol of alkylene oxide,
b1.2) (poly)alkylphenols or (poly)alkylphenol alkoxylates [=polyalkylphenol (poly)alkylene glycol ethers], for example having 1 to 12 carbon atoms per alkyl radical and 1 to 150 alkyleneoxy units in the polyalkyleneoxy moiety, preferably tri-n-butylphenol or triisobutylphenol reacted with 1 to 50 mol of ethylene oxide,
b1.3) polyarylphenols or polyarylphenol alkoxylates [=polyarylphenol (poly)alkylene glycol ethers], for example tristyrylphenol polyalkylene glycol ethers having 1 to 150 alkyleneoxy units in the polyalkyleneoxy moiety, preferably tristyrylphenol reacted with 1 to 50 mol of ethylene oxide,
b1.4) compounds which formally represent the reaction products of the molecules described under b1.1) to b1.3) with sulfuric acid or phosphoric acid, and their salts neutralized with suitable bases, for example the acidic phosphoric ester of triethoxylated phenol, the acidic phosphoric ester of a nonylphenol reacted with 9 mol of ethylene oxide and the phosphoric ester, neutralized with triethanolamine, of the reaction product of 20 mol of ethylene oxide and 1 mol of tristyrylphenol, and
b1.5) (poly)alkyl- and (poly)arylbenzenesulfonates which are acidic and have been neutralized with suitable bases, for example having 1 to 12 carbon atoms per alkyl radical or having up to 3 styrene units in the polyaryl radical, preferably (linear) dodecylbenzenesulfonic acid and its oil-soluble salts, for example the isopropylammonium salt of dodecylbenzenesulfonic acid.

The alkyleneoxy units are preferably ethyleneoxy, propyleneoxy and butyleneoxy units, in particular ethyleneoxy units.

Preferred surfactants from the group of aromatic-based surfactants are, in particular, for example

phenol reacted with 4 to 10 mol of ethylene oxide, available commercially, for example, in the form of Agrisol® products (Akcros),

triisobutylphenol reacted with 4 to 50 mol of ethylene oxide, available commercially, for example, in the form of the Sapogenat T® products (Clariant),

nonylphenol reacted with 4 to 50 mol of ethylene oxide, available commercially, for example, in the form of the Arkopal® products (Clariant),

tristyrylphenol reacted with 4 to 150 mol of ethylene oxide, for example Soprophor CY/8° (Rhodia) and

acidic (linear) dodecylbenzenesulfonate, available commercially, for example, in the form of the Marlon® products (Hüls).

Organic phosphates or phosphonates [component (b2)] for the purposes of the invention are completely reacted, unsaponified esters of orthophosphoric acid or of an alkyl-, aryl-, alkylaryl-, poly(alkyl)aryl- or poly(arylalkyl)arylphosphonic acid. Preferred here are compounds which (as far as possible) are polar, but which at the same time are largely water-insoluble and which, because of their interface activity, lower the interfacial tension of the oil droplets in the spray mixture which contain the active ingredients (a) or the active ingredient (a) relative to the external aqueous phase such that, in conjunction with the surfactants/emulsifiers additionally present in the formulation, a stable dilution/spray mixture forms which is faultless with regard to application technology. Particularly preferably suitable are compounds of the abovementioned type which have been alkoxylated before or after the esterification with orthophosphoric acid or phosphonic acid, in particular tri(butoxyethyl) phosphate (TBEP), which has a solubility in water of 1.1 g/l at 20° C.

The compounds of component (b2) have the common feature that, in aqueous solution, they do not form micellar aggregates which can be detected, for example, using light scattering measurements or other methods. This delimits them from the phosphoric ester surfactants and justifies their classification as solvents.

Suitable polar and also largely water-insoluble organic phosphoric esters are the esters of orthophosphoric acid which have been formally reacted three times with alcohols, and the oxalkylates of orthophosphoric acid reacted formally once and/or twice with alcohols. Examples of suitable compounds in this connection are:
b2.1) largely water-insoluble polar esters of phosphoric acid with alcohols from the group comprising phosphoric esters with
   monohydric alkanols having 5 to 22 carbon atoms, e.g. with n-, i- or neopentanol, n-hexanol, n-octanol, 2-ethylhexanol,
   diols or polyols, such as ethylene glycol, propylene glycol or glycerol,
   aryl, alkylaryl, poly(alkyl)aryl and poly(arylalkyl)aryl alcohols, for example with phenol, cresol, octylphenol, nonylphenol, triisobutylphenol and/or tristyrylphenol,
   alkoxylated alcohols obtained by reacting the abovementioned alcohols with alkylene oxides, preferably (C₁-C₄)alkylene oxides, and
   alkoxylated alcohols obtained by reacting monohydric alkanols having 1 to 4 carbon atoms and alkylene oxides, where the 3 alcohol components of the phosphoric ester may be identical or different and are chosen such that the ester can be used as a largely water-insoluble polar solvent.
      Also suitable are
b2.2) largely water-insoluble and also polar phosphonates based on alkyl-, aryl-, alkylaryl-, poly(alkyl)aryl- or poly(arylakyl)arylphosphonic acids, diesterified with alcohols and/or alkoxylated alcohols, preferably esters with
   monohydric alkanols having 1 to 22 carbon atoms, e.g. with e.g. n-methanol, n-ethanol, n- or i-propanol, n-, i- or t-butanol, n-, i- or neopentanol, n-hexanol, n-octanol, 2-ethylhexanol, or else sec-butanol,
   diols or polyols, such as ethylene glycol, propylene glycol or glycerol, aryl, alkylaryl, poly(alkyl)aryl or poly(arylalkyl)aryl alcohols, for example with phenol, cresol, octylphenol, nonylphenol, triisobutylphenol and/or tristyrylphenol or
   alkoxylated alcohols obtained by reacting the abovementioned alcohols with alkylene oxides, preferably (C₁-C₄)alkylene oxides,
      as the respective alcohol component, where the 2 alcohol components of the phosphonic ester may be identical or different and are chosen such that the ester can be used as a largely water-insoluble polar solvent.

In principle, the alkyleneoxy units are preferably (C₁-C₄)alkylene oxide units, e.g. ethyleneoxy, propyleneoxy and/or butyleneoxy units, in particular propyleneoxy and/or ethyleneoxy units.

The alcohol components preferably contain 1-200, in particular 1-150, very particularly 1-100 alkyleneoxy units, preferably ethyleneoxy units.

Preferred phosphoric esters are, in particular, for example,
orthophosphoric acid triesterified with alkoxylated short-chain alcohols having 1 to 22 carbon atoms in the alkyl radical and 1 to 30 alkyleneoxy units in the polyalkyleneoxy moiety, for example tributoxyethyl phosphate (Clariant),
orthophosphoric acid triesterified with alkyl alcohols having 5 to 22 carbon atoms, for example Hostaphat CG 120° (Clariant), tri-n-octyl phosphate (“TOF”, Bayer), and
orthophosphoric acid partially esterified with optionally alkoxylated alcohols having 1 to 22 carbon atoms in the alkyl moiety or optionally alkoxylated phenol derivatives, in each case having 0 to 30 alkyleneoxy units in the polyalkyleneoxy moiety, where the remaining OH valences of the orthophosphoric acid have been subsequently alkoxylated (e.g. with 1 to 10 mol of alkylene oxide having 1 to 4 carbon atoms), for example the reaction product of mono-/dibutoxyethyl phosphate and 2 mol of ethylene oxide or 2 mol of propylene oxide (Clariant).

Preferred phosphonates are, in particular, for example esters of n-octylphosphonic acid reacted formally twice with alcohols, for example the Hostarex grades® (Clariant).

In addition, the formulations according to the invention comprise further solvents, surfactants and/or polymers without said advantageous properties of the surfactant/solvent system being lost. Optionally, it is also possible, for example, to incorporate anionogenic surfactants, such as alkyl polyglycol ether carboxylates, into the formulations. Examples of such anionogenic surfactants are Akypo RLM 45® (Kao) and Marlowet 4538® (Condea).

Likewise, cationic or other nonionogenic surfactants can also be incorporated into the emulsifiable concentrates according to the invention. Examples of cationogenic surfactants are Genamin C-200® (Clariant) and Armoblen 557® (Akzo), and examples of suitable nonionogenic surfactants are Emulsogen EL 400® (Clariant), Serdox NOG 600® (Servo) or also surface-active polymers based on alkylene oxide, such as, for example, ethylene oxide/propylene oxide block copolymers (e.g. Genapol PF40® (Clariant)).

In conjunction with the present invention, suitable additional solvents are, for example, nonpolar solvents, polar protic or aprotic dipolar solvents and mixtures thereof. Examples of solvents for the purposes of the invention are
aliphatic or aromatic hydrocarbons, such as, for example, mineral oils, paraffins or toluene, xylenes and naphthalene derivatives, in particular 1-methylnaphthalene, 2-methylnaphthalene, 6-16C-aromatic mixtures, such as, for example, the Solvesso® series (ESSO) with the grades Solvesso® 100 (b.p. 162-177° C), Solvesso® 150 (b.p. 187-207° C.) and Solvesso® 200 (b.p. 219-282° C.) and 6-20C-aliphatics, which may be linear or cyclic, such as the products of the Shellsol® series, grades T and K or BP-n paraffins,
halogenated aliphatics or aromatic hydrocarbons, such as methylene chloride or chlorobenzene,
esters, such as, for example, triacetin (acetic triglyceride), butyrolactone, propylene carbonate, triethyl citrate and (C₁-C₂₂)alkyl phthalates, specifically (C₄-C₈)alkyl phthalates,
ethers, such as diethyl ether, tetrahydrofuran (THF), dioxane, alkylene glycol monoalkyl ethers and dialkyl ethers, such as, for example, propylene glycol monomethyl ether, specifically Dowanol® PM (propylene glycol monomethyl ether), propylene glycol monoethyl ether, ethylene glycol monomethyl ether or monoethyl ether, diglyme and tetraglyme,
amides, such as dimethylformamide (DMF), dimethylacetamide, dimethylcaprylic/capric fatty acid amide and N-alkylpyrrolidones,
ketones, such as water-soluble acetone, but also water-immiscible ketones, such as, for example, cyclohexanone or isophorone,
nitriles, such as acetonitrile, propionitrile, butyronitrile and benzonitrile,
sulfoxides and sulfones such as dimethyl sulfoxide (DMSO) and sulfolane, and
oils in general, e.g. vegetable-based oils, such as com oil and rapeseed oil.

Also frequently suitable are combinations of different solvents which additionally comprise alcohols such as methanol, ethanol, n- and i-propanol, n-, i-, t- and 2-butanol.

Preferred additional organic solvents for the purposes of the present invention are, in particular, amides, such as dimethylcaprylic/capric fatty acid amide and N-methylpyrrolidone.

Using the surfactant/solvent systems according to the invention it is now surprisingly possible to prepare optically transparent, thermodynamically stable and liquid emulsifiable concentrates inter alia of biscarbamate herbicides (desmedipham and/or phenmedipham) and/or sulfonate herbicides (ethofumesate). Moreover, the surfactant system according to the invention influences the pesticidal action of the incorporated active ingredient(s) in a favorable manner.

The surfactant/solvent system according to the invention also permits the preparation of emulsifiable concentrates with active ingredients other than those listed here, provided they have similar properties with regard to their solubilities. Suitable examples are also herbicides from the group of phenoxyphenoxypropionates, such as diclofop-methyl, cyhalofop-butynyl, of heteroaryloxyphenoxypropionates, such as fenoxaprop-ethyl, fenoxaprop-P-ethyl, fluazifop-butyl, fluazifop-P-butyl, haloxyfop-methyl, haloxyfop-etotyl, haloxyfop-P-methyl, propquizofop, quizalofop-ethyl, quizalofop-P-ethyl or clodinafop-propargyl, from the group of triazinones, such as metamitron, metribuzin or hexazinone, from the group of sulfonyureas, such as triflusulfuron-methyl, amidosulfuron, iodosulfuron-methyl, tribenuron-methyl, triasulfuron, thifensulfuron-methyl, sulfosulfuron, sulfometuron-methyl, prosulfuron, primisulfuron-methyl, oxasulfuron, metsulfuron-methyl, ethoxysulfuron, ethametsulfuron-methyl, cyclosulfamuron, cinosulfuron, chlorsulfuron, chlorimuron-ethyl or bensulfuron-methyl, preferably in the form of the nonsalts, but also slightly water-soluble pyridylsulfonylureas, or other herbicides, such as benfuresates, or other active ingredients, such as the fungicide such as prochloraz and/or insecticides such as deltamethrin. This demonstrates the flexibility of the described surfactant/solvent system. Said compounds are known from “The Pesticide Manual”, British Crop Protection Council, 11th Edition, 1997.

For the reasons given, the invention provides in particular surfactant/solvent systems for liquid herbicidal compositions which comprise
a) one or more biscarbamate herbicides of the formulae (a1) and (a2) 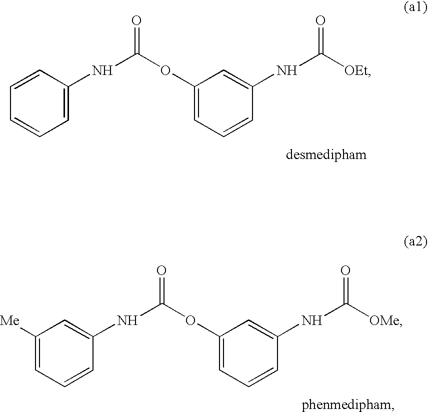
   and/or one or more herbicides of the sulfonate type, such as, for example, ethofumesates (a3), 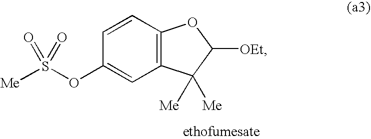
b) the surfactant/solvent system according to the invention (component mixture (b)),
c) optionally further organic solvents and
d) optionally further surfactants and/or polymers.

The compounds of the formulae (a1) and (a2) are derivatives of carbamic acid. The herbicidal properties of these compounds are described, for example, in DE-A-3799758.

The compounds of the formula (a3) contain an asymmetrical carbon atom. Both enantiomers are regarded as biologically active. The formula (a3) thus covers all stereoisomers and mixtures thereof, in particular the racemate. Their herbicidal properties are described, for example, in GB-A-1271659.

The surfactant/solvent system according to the invention (component mixture b)) gives, upon dilution with water, dispersions of oil phases in water or, in the case of appropriate selection of the individual components, of aqueous phases in oil. Depending on the composition, dispersions are therefore accessible which are dilutable either with water or with oil with retention of the colloidal structure as a result. The dispersions accessible via dilution from the concentrates described are therefore further provided by the invention.

The weight ratios of the combined herbicidal active ingredients of type a) (desmedipham(a1):phenmedipham(a2):ethofumesates(a3)) can vary within wide limits and are usually between 1:1:1 and 1:10:100, in the case of pure biscarbamate mixtures (a1):(a2) between 100:1 and 1:100. For mixtures with all three herbicidal active ingredients, the following (a1):(a2) (a3) weight ratios are particularly preferred:
(a1):(a2):(a3) as 1:1:1 to 1:2:3, in particular 1:1.2:1.4 to 1:1.8:2.4 and
(a1):(a2):(a3) as 1:2:5 to 1:5:10, in particular 1:2.5:5.5 to 1:3.5:6.5.

The application amounts are usually between 400 and 2000 g a.i./ha, preferably between 600 and 1500 g a.i./ha. For the same herbicidal action, the application amount in the case of the combined use of all three herbicides (a1)-(a3) is considerably below the application amounts for the application of combinations or individual applications of the biscarbamate herbicides of the type (a1) and (a2): for example, the application amount in the case of pure biscarbamate mixtures (a1):a2) is between 600 and 1300 g a.i./ha, in the case of three-component mixtures a1):a2):a3) it is between 400 and 1000 g a.i./ha. For this reason, thermodynamically stable formulations in which all three active ingredients are present are of particular interest because of their high biological effectiveness for an overall reduced active ingredient content. However, the optimal choice of the weight ratios and of the application amounts is dependent on the development stage of the respective weeds or weed grasses, the prevailing weed spectra, environmental factors and climatic conditions, meaning that the weight ratios and application amounts given above are to be checked in individual cases.

The auxiliaries necessary for the preparation of said formulations, such as, in particular, surfactants and solvents, are known in principle and are described, for example, in: McCutcheon's “Detergents and Emulsifiers Annual”, MC Publ. Corp., Ridgewood N.J.; Sisley and Wood, “Encyclopedia of Surface active Agents”, Chem. Publ. Co. Inc., N.Y. 1964; Schönfeldt, “Grenzflächenaktive Äthylenoxidaddukte” [Surface-active ethylene oxide adducts], Wiss. Verlagsgesellschaft, Stuttgart 1976; Winnacker-Küchler, “Chemische Technologie” [Chemical Engineering], Volume 7, C. Hauser-Verlag, Munich, 4th Edition 1986.

While the chemical “structure” of the individual components which can be used is sufficiently described therein, predictions with regard to the properties of mixtures of such components for the formulation of a certain active ingredient system cannot usually be deduced from said handbooks. If, for example, a surfactant/solvent combination is used which results in stable emulsifiable concentrates for active ingredient combinations of desmedipham and/or phenmedipham and/or ethofumesate at a relatively low “active ingredient loading”, then, despite the presence of aromatic-based surfactants, if the active ingredient concentration is increased, stable emulsifiable concentrates are no longer obtained, but unstable multiphase systems, as Table 1 (see below) shows (Example 1). This gives rise directly to the importance of the solvent component according to the invention (orthophosphoric esters and/or phosphonic esters) for the stability of the emulsifiable concentrates to be prepared.

However, the aromatic character of the surfactants used is also of equal importance. This is clear from Examples 2 and 3: if a (conventional) aliphatic hydrocarbon surfactant is used instead of an aromatic-based surfactant, unstable emulsifiable concentrates are likewise obtained. This underlines the fact that the aromatic-based surfactant component is an essential constituent of the described invention.

In Example 4, the “limiting concentration” of ethofumesate is exceeded, leading to crystallization of the active ingredient. Replacing the tributyl phosphate (TBP) used as solvent in formulation 4 by tributoxyethyl phosphate (TBEP) gives, in contrast, a stable emulsifiable concentrate (Example XVII). TBEP thus permits a higher loading of the formulation with, in particular, ethofumesate and thus surpasses TBP with regard to “solubilizing power”. Irrespective thereof, in the individual case, however, further solvents may also be required in order to obtain a stable, crystal-free emulsifiable concentrate.

Starting from these component mixtures, which do not contain stable emulsifiable concentrates of active ingredients (a1), (a2) and/or (a3), in particular, it was not to be expected as a result that stable emulsifiable concentrates can be prepared for, in particular, the active ingredients described under (a1)-(a3) using the surfactant/solvent system according to the invention.

Surprisingly, it has now been found that combinations of one orthophosphoric ester and/or one phosphonic ester as solvent and one or more aromatic-based surfactants are particularly well suited for the preparation of stable emulsifiable concentrates. This becomes clear by reference to the examples listed in Table 2 (see below), which give an idea of the chemical flexibility of the disclosed component mixture. Thus, suitable aromatic-based surfactants are, in particular, alkoxylates of triisobutylphenol, where the degree of ethoxylation should preferably be 4-10 mol of EO, in particular 6-8 mol of EO, per molecule (Examples I and VII) (EO=ethyleneoxy).

A critical factor for the choice of further surfactant components is their “acidity or basicity per weight or volume unit”, which is expressed by the acid number or amine number. Too great an increase in the overall. acid number or amine number in the finished formulation is problematical in as much as it causes flocculation/crystallization of the formulated active ingredient(s). Therefore, when choosing further surfactant components, it must be taken into consideration that the overall acid number or amine number does not increase too greatly. Consequently, as well as nonionogenic surfactants, only acidic or basic components with a sufficiently low acid number or amine number are preferably suitable. Since said number correlates in turn with the molecular weight, suitable further surfactant components are often also acidic or basic derivatives of compounds with a high molecular weight, such as, for example, tristyrylphenol alkoxylates (Example XI). In the case of nonionogenic surfactants, these considerations, corresponding to the nonionogenic character of these components, are invalid. Accordingly, additional nonionogenic surfactant components which may be used, as Examples VIII, IX and X show, are, as well as castor oil reacted with 40 mol of EO, in particular, for example, also castor oil reacted with only 12 mol of EO, oleic acid reacted with 15 mol of EO and EO-PO-EO block copolymers. In addition, Example XII shows that phosphonates can also be used instead of the orthophosphoric esters as solvents.

In this connection, it is also to be pointed out that the surfactant/solvent systems described permit the preparation of stable emulsifiable concentrates with an active ingredient loading and composition which is variable within wide limits (Examples I-VII): thus, for example, the active ingredient loading can vary between 20 and 40, preferably between 24 and 30, percent by weight (Examples I-IV). With regard to the active ingredient composition, using this component mixture, not only are stable “single active ingredient emulsifiable concentrates” accessible, but also those with two or, in particular, three active ingredients, preferably of the type a1), a2) and/or a3) (Examples II, V and VI).

In addition, Examples XIII, XIV, XV and XVI show that the surfactant/solvent systems described are also suitable for the preparation of emulsifiable concentrates with active ingredients other than those explicitly listed under (a1)-(a3). Thus, for example, they can also be used to prepare emulsifiable concentrates which contain the active ingredients diclofop-methyl, fenoxaprop-ethyl, prochloraz and/or deltamethrin.

Preferred weight ratios of the components orthophosphoric ester/phosphonic ester:aromatic-based surfactant are, depending on the active ingredient loading and composition of the emulsifiable concentrates, 100:1 to 1:100, particularly preferably 20:1 to 1:20, further preferably 5:1 to 1:2, for example 1.5:1 to 1.3:1.

Emulsifiable concentrates which are prepared in accordance with the present invention comprise a priori no additional water, but only the residual water present in said commercially available surfactants and surfactant mixtures, polymers and solvents. However, because of the surfactants present in the formulations, it is possible to dilute said formulations with water up to a critical volume fraction without the formulation becoming cloudy or unstable. This gives rise formally firstly to W/O microemulsions which, upon further increasing the water fraction, convert to W/O emulsions and finally, upon further dilution with water, to O/w emulsions. The invention thus also covers liquid formulations of, in particular, one or more active ingredients of the type (a1), (a2) and/or (a3) which, in addition to the surfactant/solvent mixture b) according to the invention, also comprise (additional) water.

Using the component mixtures (b), it is possible to prepare, preferably, liquid formulations, e.g. including emulsifiable concentrates, in particular preparations of des- and/or phenmedipham and/or ethofumesates, characterized by a content of
a) 1 to 50% by weight, preferably 15 to 35% by weight, of pesticidal active ingredients,
b) 5 to 80% by weight, preferably 10 to 70% by weight, of the surfactant/solvent system (b) according to the invention,
c) 0 to 40% by weight, preferably 5 to 35% by weight, of further organic solvents,
d) 0 to 30% by weight, preferably 10 to 25% by weight, of further surfactants,
e) 0 to 20% by weight, preferably 0 to 10% by weight, of customary formulation auxiliaries and
f) 0 to 96% by weight, preferably 0 to 90% by weight, in particular 0 to 10% by weight, of water.

Largely anhydrous emulsifiable concentrates represent a favorable application form of the herbicidal active ingredients of type (a) and are a preferred subject-matter of the invention. Particular preference is given to emulsifiable concentrates with a content of
a) 10 to 40% by weight of active ingredient of said type (a), preferably (a1), (a2) and/or (a3),
b) 10 to 60% by weight of the surfactant/solvent system (b) according to the invention,
c) 5 to 35% by weight of further organic solvents,
d) 10 to 25% by weight of further surfactants and
e) 0 to 10% by weight of customary formulation auxiliaries.

Customary formulation auxiliaries e) are, for example, antifreeze agents, evaporation inhibitors, preservatives, fragrances, dyes, inter alia; preferred formulation auxiliaries e) are
antifreeze agents and evaporation inhibitors such as glycerol, e.g. in an amount of from 2 to 10% by weight and
preservatives, e.g. Mergal K9N® (Riedel) or Cobate C®, in the customary use concentrations for the compositions used specifically in each case.

The formulations and: spray mixtures prepared using the surfactant/solvent system according to the invention also give results which are advantageous from a biological viewpoint upon use. For example, it is observed that the biological activity of the pesticidal active ingredients used can be increased in a synergistic manner by the use of component (b) according to the invention.

In the examples below, the quantities are based on weight, unless stated otherwise. The examples in Table 1 refer to comparative examples which are not in accordance with the invention, while those in Table 2 are in accordance with the invention.

TABLE 1
Examples of formulations which do not give stable emulsifiable
concentrates (EC)

| | 1 | 2 | 3 | 4 |
|---|---|---|---|---|
| Desmedipham (a1) | 8 | 2.75 | 2.75 | |
| Phenmedipham (a2) | 10 | 8.25 | 8.25 | |
| Ethofumesate (a3) | 12.5 | 16.5 | 16.5 | 37.25 |
| Rapeseed oil | 18 | | | |
| C₇H₁₅/C₉H₁₉—CON(CH₃)₂¹⁾ | 23 | | | |
| [C₄H₉—O]₃-PO²⁾ | | | | 21.55 |
| [C₄H₉—O-(EO)]₃-PO³⁾ | | 28.5 | 28.5 | |
| N-Methylpyrrolidone (NMP) | 8.5 | | | |
| [NP-O-(EO)₆-]ₙ-PO₄₋ₙH₃₋ₙ⁴⁾ | 2 | | | |
| [(s-C₄H₉)₃—C₆H₂]—O-(EO)₆-H⁵⁾ | 18 | | | 18.6 |
| C_{12/14}—O-(EO)₂₃⁶⁾ | | 20 | 20 | |
| Castor oil + 40 mol of EO⁷⁾ | | 18 | | |
| EO-PO-EO block cop., 80% EO⁸⁾ | | | 18 | |
| [C₆H₅—O-(EO)₄]ₙ-PO₄₋ₙH₃₋ₙ⁹⁾ | | 4 | 4 | 3.9 |
| [i-C₁₃—O-(EO)₂₀-]ₙ-PO₄₋ₙH₃₋ₙ¹⁰⁾ | | 2 | 2 | 2 |

Abbreviations and footnotes in Table 1: see after Table 2

Abbreviations and footnotes in Table 1: see after Table 2

TABLE 2
Formulation examples for emulsifiable concentrates according to the invention

| | I | II | III | IV | V | VI | VII | VIII | IX | X | XI | XII | XIII | XIV | XV | XVI | XVII |
|---|---|---|---|---|---|---|---|---|---|---|---|---|---|---|---|---|---|
| Desmedipham (a1) | 6.5 | 8 | 2.5 | 2.75 | 32 | 16 | 6.5 | 6.5 | 6.5 | 6.5 | 6.5 | 6.5 | | | | | |
| Phenmedipham (a2) | 8.5 | 10 | 7.25 | 8.25 | | 16 | 8.5 | 8.5 | 8.5 | 8.5 | 8.5 | 8.5 | | | | | |
| Ethofumesate (a3) | 10.5 | 12.5 | 14.75 | 16.5 | | | 10.5 | 10.5 | 10.5 | 10.5 | 10.5 | 10.5 | | | | | 37.25 |
| Diclofop-methyl | | | | | | | | | | | | | 15 | | | | |
| Fenoxaprop-ethyl | | | | | | | | | | | | | | 15 | | | |
| Prochloraz | | | | | | | | | | | | | | | 40 | | |
| Deltamethrin | | | | | | | | | | | | | | | | 5 | |
| [C₄H₉—O-(EO)]₃-PO³⁾ | 29 | 25.5 | 30 | 28.5 | 2 | 2 | 29 | 29 | 29 | 29 | 29 | | 40 | 40 | 27 | 30 | 21.55 |
| [(s-C₄H₉)₃—C₆H₂]—O- | 20 | 20 | 20 | 20 | 20 | 20 | | 20 | 20 | 20 | 20 | 20 | 20 | 20 | 15 | 30 | 18.6 |
| (EO)₆-H⁵⁾ | | | | | | | | | | | | | | | | | |
| [(s-C₄H₉)₃—C₆H₂]—O- | | | | | | | 20 | | | | | | | | | | |
| (EO)₈-H¹¹⁾ | | | | | | | | | | | | | | | | | |
| Castor oil + 40 mol of EO⁷⁾ | 19 | 18 | 19 | 18 | | | 19 | | | | | 19 | 19 | 19 | 14 | 25 | 16.7 |
| Castor oil + 12 mol of EO¹²⁾ | | | | | | | | 19 | | | | | | | | | |
| Oleic acid + 15 mol of EO¹³⁾ | | | | | | | | | 19 | | | | | | | | |
| EO-PO-EO block | | | | | 18 | 18 | | | | 19 | | | | | | | |
| cop., 80% EO⁸⁾ | | | | | | | | | | | | | | | | | |
| [(Tri-sty)-Phe-O-(EO)₁₆]ₙ- | | | | | | | | | | | 19 | | | | | | |
| PO₄₋ₙH₃₋ₙ¹⁴⁾ | | | | | | | | | | | | | | | | | |
| [C₆H₅—O-(EO)₄]ₙ-PO₄₋ₙH₃₋ₙ⁹⁾ | 4.5 | 4 | 4.5 | 4 | 4 | 4 | 4.5 | 4.5 | 4.5 | 4.5 | 4.5 | 4.5 | 4 | 4 | 4 | 10 | 3.9 |
| [i-C₁₃—O-(EO)₂₀-]ₙ- | 2 | 2 | 2 | 2 | 2 | 2 | 2 | 2 | 2 | 2 | 2 | 2 | 2 | 2 | | | 2 |
| PO₄₋ₙH₃₋ₙ¹⁰⁾ | | | | | | | | | | | | | | | | | |
| N-Methylpyrrolidone (NMP) | | | | | 8 | 8 | | | | | | | | | | | |
| C₇H₁₅/C₉H₁₉—CON(CH₃)₂¹⁾ | | | | | 14 | 14 | | | | | | | | | | | |
| C₈H₁₇—PO(—O-2- | | | | | | | | | | | | 29 | | | | | |
| Ethylhexyl)₂¹⁵⁾ | | | | | | | | | | | | | | | | | |

Abbreviations and footnotes to Tables 1 and 2:
n In the formulae, n=0-3, i.e. in each case it is a mixture of phosphoric esters where n=1, 2 and 3, the acidic fractions where n=1 and 2 being essential;
numerals all data are proportions in percent by weight based on the weight of the formulation (=100 percent by weight);
i-C₁₃—=isotridecyl
EO=“ethylene oxide”, i.e. a group of the formula —CH₂—CH₂—O— (ethyleneoxy) or, if terminal, —CH₂—CH₂—O—H (hydroxyethyl)
PO=“propylene oxide”, i.e. a group of the formula —C₃H₆—O— (propyleneoxy)
(Tri-sty-)Phe-=tristyrylphenyl-
NP—=nonylphenyl
   Footnotes to Tables 1 and 2 (explanations of-index numbers):
1) Caprylic/capric fatty acid dimethylamide (specifically Genagen 4166®, Clariant, or Hallcomid M 8-10®, Hall Chemicals)
2) Tributyl phosphate (specifically Antifoam T®, Bayer)
3) Tributoxyethyl phosphate (specifically Hostaphat B310®, Clariant)
4). Phosphated nonylphenol alkoxylate (specifically Emcol CS 136®, Witco)
5) Ethoxylated tri(sec-butyl)phenol (specifically Sapogenat T-060®, Clariant)
6) Ethoxylated C_{12/14}-fatty alcohol (specifically Brij 35®, ICI)
7) Ethoxylated castor oil (specifically Emulsogen EL 400®, Clariant)
8) Ethylene oxide-propylene oxide-ethylene oxide block copolymer (specifically Genapol PF 80®, Clariant)
9) Phosphated ethoxylated phenol with the following proportions in the mixture:
   7.5-8.5% by weight n=0, 1-10% by weight C₆H₅—O-(EO)₄H, 80-90% by weight n=1 and about 2%. by weight n=2
10) Phosphated ethoxylated isotridecyl alcohol (specifically Servoxyl VPDZ 20/100®, Servo)
11 ) Ethoxylated tri(sec-butyl)phenol (specifically Sapogenat T-080®, Clariant)
12) Ethoxylated castor oil (specifically Etocas 12®, Croda)
13) Ethoxylated oleic acid (specifically Serdox NOG-600®, Servo)
14) Phosphated tristyrylphenol alkoxylate (specifically Soprophor 3D33®, Rhodia)
15) Diester of octanephosphonic acid (specifically Hostarex PO 224®, Clariant)

1-13. (canceled).

14. A surfactant/solvent system for liquid organic formulations, comprising:
b1) one or more aromatic-based surfactants; and
b2) one or more completely esterified organic phosphates and/or phosphonates as solvent, selected from the group consisting of
b2.1) esters of phosphoric acid with alcohols selected from the group consisting of
monohydric alkanols having 5 to 22 carbon atoms,
diols or polyols,
aryl, alkylaryl, poly(alkyl)aryl or poly(arylalkyl)aryl alcohols,
alkoxylated alcohols obtained by reacting the abovementioned alcohols with alkylene oxides, or
alkoxylated alcohols obtained by reacting monohydric alkanols with 1 to 4 carbon atoms and alkylene oxides,
b2.2) phosphonates based on alkyl-, aryl-, alkylaryl-, poly(alkyl)aryl- or poly(arylalkyl)arylphosphonic acids diesterified with alcohols and/or alkoxylated alcohols selected from the group consisting of
monohydric alkanols having 1 to 22 carbon atoms,
diols or polyols,
aryl, alkylaryl, poly(alkyl)aryl and poly(arylalkyl)aryl alcohols or
alkoxylated alcohols obtained by reacting the abovementioned alcohols with alkylene oxides,
wherein the 3 alcohol components of the phosphoric ester (b2.1) or the two alcohol components of the phosphonate (b2.2) can be identical or different and are chosen such that the ester is a polar solvent which is water-insoluble or soluble in water to 5 g/l at 20° C. and, in aqueous solution, does not form micellar aggregates.

15. The surfactant/solvent system as claimed in claim 14, wherein said one or more aromatic-based surfactants are selected from the group consisting of:
b1.1) phenols, phenyl (C₁-C₄)alkyl ethers or (poly)alkoxylated phenols,
b1.2) (poly)alkylphenols or (poly)alkylphenol alkoxylates,
b1.3) polyarylphenols or polyarylphenol alkoxylates,
b1.4) compounds which formally represent the reaction products of the molecules described under b1.1) to b1.3) with sulfuric acid or phosphoric acid, and their salts neutralized with suitable bases,
b1.5) (poly)alkyl- and (poly)arylbenzenesulfonates which are acidic and have been neutralized with suitable bases.

16. The surfactant/solvent system as claimed in claim 14, wherein said one or more aromatic-based surfactants are selected from the group consisting of:
phenol reacted with 4 to 10 mol of ethylene oxide,
triisobutylphenol reacted with 4 to 50 mol of ethylene oxide,
nonylphenol reacted with 4 to 50 mol of ethylene oxide,
tristyrylphenol reacted with 4 to 150 mol of ethylene oxide, and
acidic (linear) dodecylbenzenesulfonate.

17. The surfactant/solvent system as claimed in claim 14, further comprising one or more compounds selected from the group consisting of:
orthophosphoric acid triesterified with alkoxylated short-chain alcohols having 1 to 22 carbon atoms in the alkyl radical and 1 to 30 alkyleneoxy units in the polyalkyleneoxy moiety,
orthophosphoric acid triesterified with alkyl alcohols having 5 to 22 carbon atoms,
orthophosphoric acid partially esterified with optionally alkoxylated alcohols having 1 to 22 carbon atoms in the alkyl radical or optionally alkoxylated phenol derivatives, in each case having 0 to 30 alkyleneoxy units in the polyalkyleneoxy moiety, the remaining OH valences of the orthophosphoric acid having been subsequently alkoxylated, and
esters of n-octylphosphonic acid which have been formally reacted twice with alcohols.

18. The surfactant/solvent system as claimed in claim 14, comprising tri(butoxyethyl) phosphate as solvent component (b2).

19. The surfactant/solvent system as claimed in claim 15, comprising tri(butoxyethyl) phosphate as solvent component (b2).

20. The surfactant/solvent system as claimed in claim 15, comprising tri(butoxyethyl) phosphate as solvent component (b2).

21. A liquid formulation, comprising:
(a) one or more water-insoluble active ingredients;
(b) the surfactant/solvent system as defined in claim 14, wherein component mixture (b) comprises b1) and b2);
(c) optionally further organic solvents;
(d) optionally further surfactants and/or polymers; and
(e) optionally water.

22. The liquid formulation as claimed in claim 21, comprising
(a) 1 to 50% by weight of pesticide active ingredients;
(b) 5 to 80% by weight of the component mixture (b);
(c) 0 to 40% by weight of further organic solvents;
(d) 0 to 30% by weight of further surfactants;
(e) 0 to 20% by weight of customary formulation auxiliaries; and
(f) 0 to 96% by weight of water.

23. The liquid formulation as claimed in claim 21, comprising tri(butoxyethyl) phosphate as solvent component (b2).

24. The liquid formulation as claimed in claim 22, comprising tri(butoxyethyl) phosphate as solvent component (b2).

25. The liquid formulation as claimed in claim 21, further comprising one or more active ingredients selected from the group consisting of the herbicides desmedipham, phenmedipham and ethofumesate.

26. The liquid formulation as claimed in claim 22, further comprising one or more active ingredients selected from the group consisting of the herbicides desmedipham, phenmedipham and ethofumesate.

27. The liquid formulation as claimed in claim 23, further comprising one or more active ingredients selected from the group consisting of the herbicides desmedipham, phenmedipham and ethofumesate.

28. The liquid formulation as claimed in claim 24, further comprising one or more active ingredients selected from the group consisting of the herbicides desmedipham, phenmedipham and ethofumesate.

29. An emulsifiable concentrate, comprising:
(a) 10 to 40% by weight of one or more water-insoluble active ingredients;
(b) 10 to 60% by weight of the surfactant/solvent system as defined in claim 14, wherein component mixture (b) comprises b1) and b2);
(c) 5 to 35% by weight of further organic solvents;
(d) 10 to 25% by weight of further surfactants; and
(e) 0 to 10% by weight of customary formulation auxiliaries.

30. The emulsifiable concentrate as claimed in claim 29, comprising tri(butoxyethyl) phosphate as solvent component (b2).

31. The liquid formulation as claimed in claim 29, further comprising one or more active ingredients selected from the group consisting of the herbicides desmedipham, phenmedipham and ethofumesate.

32. The liquid formulation as claimed in claim 30, further comprising one or more active ingredients selected from the group consisting of the herbicides desmedipham, phenmedipham and ethofumesate.

33. A process for the preparation of a formulation as defined in claim 21, comprising the step of mixing the components with one another.

34. A method of controlling undesired plant growth, comprising the step of applying an effective amount of a formulation as defined in claim 21, wherein said formulation comprises a herbicidal active ingredient, to plants, plant parts or an area where the plants grow, wherein said formulation is optionally diluted with water prior to application.

35. A method of controlling undesired plant growth, comprising the step of applying an effective amount of a formulation as defined in claim 29, wherein said formulation comprises a herbicidal active ingredient, to the plants, plant parts or area where the plants grow, wherein said formulation is diluted with water prior to application.
